(19) United States
(12) Patent Application Publication
(10) Pub. No.: US 2004/0220656 A1
(43) Pub. Date: Nov. 4, 2004

(54) Coated medical devices and methods of making the same

(76) Inventors: Samuel J. Epstein, Newton, MA (US); Wendy Naimark, Cambridge, MA (US)

Correspondence Address: JONES DAY, 222 EAST 41ST ST, NEW YORK, NY, 10017, US

(21) Appl. No.: 10/426,642

(22) Filed: Apr. 30, 2003

(51) Int. Cl. A61F 2/06

(52) U.S. Cl. 623/1.15; 623/1.42

(57) ABSTRACT

A medical device, such as a stent, for delivering a therapeutic agent to body tissue of a patient and a method of making such a medical device are disclosed. The medical device comprises a surface, a first coating layer comprising a biological activator such as a transfection agent adhering to at least a portion of the surface, and a second coating layer comprising a therapeutic agent disposed over at least a portion of the first coating layer. The transfection agent enhances the delivery of the therapeutic agent to the targeted body tissue, and reduces the surface tension of the surface of the medical device. The transfection agent may be a poloxamer and the therapeutic agent may be a genetic material. The disclosed method allows for greater efficiency in applying the therapeutic agent onto the medical device surface and enhances delivery of the therapeutic agent to the targeted body tissue.

### FIELD OF THE INVENTION

The invention relates generally to a medical device that is useful for delivering a biologically active material to a body tissue, such as a body lumen, and a method for making such a medical device. More particularly, the invention is directed to a medical device having a surface coated with a first coating layer comprising a biological activator such as a transfection agent and a second coating layer comprising a therapeutic agent, wherein the second coating layer is disposed over the first coating layer. The transfection agent enhances the delivery of the therapeutic agent to the targeted body tissue, and reduces the surface tension of the surface of the medical device onto which the coating layer comprising the therapeutic agent is applied.

### BACKGROUND OF THE INVENTION

Medical devices, such as implanted stents, have been used for delivering therapeutic agents to body tissue such as a body lumen. These medical devices have been coated with compositions, comprising a therapeutic agent, by various methods. For example, spraying is a common technique for applying a coating uniformly to a surface of a medical device, such as a stent. Direct deposition is another method that involves depositing a bead of material along the struts of a stent.

However, many methods for coating medical devices are often inefficient because the surfaces of the medical devices tend to be hydrophobic while many therapeutic agents, such as genes, proteins, and cells, that are in an aqueous solution, have a low affinity for the relatively hydrophobic surface. Because of the surface tension between the hydrophobic surface and the aqueous solution of therapeutic agent, it is often difficult to sufficiently adhere the therapeutic agent to a medical device surface. The aqueous solution containing the therapeutic agent does not adequately wet the surface of the medical device. For example, material applied by spraying or direct deposition does not adequately wet the surface of the stent and thus does not remain on the surface. In addition, material applied by stereo lithographic application (SLA) tends to bead together and not coat a surface uniformly.

These coating techniques are also economically inefficient. Large quantities of costly therapeutic agents are wasted because it is difficult to adhere them to the surface of the medical device. The high cost of these materials coupled with the inefficiency of the coating methods make these existing methods for coating medical devices problematic. Furthermore, because it is difficult to sufficiently adhere the therapeutic agent to the medical device, it is also difficult to effectively deliver a therapeutic agent from a medical device to targeted body tissue.

To enhance delivery of the therapeutic agent from a coating layer, it may be desirable to include a biological activator, such as a transfection agent, in such coating layer. However, applying a coating layer including a therapeutic agent and a transfection agent to a medical device is often problematic. For example, it would be difficult to adhere a coating composition comprising a therapeutic agent and a transfection agent to the surface of a medical device because of the surface tension between the composition and the surface. In addition, it is desirable to prepare separate formulations of the therapeutic agent and the transfection agent that are suitable for delivery to the body tissue. Mixing the therapeutic agent and the transfection agent together in one composition would adversely affect these desired formulations. For example, mixing the separate formulations may result in inactivation of the therapeutic agent. The therapeutic agent may also be inactivated by direct exposure to the surface of the medical device. In such case, there may be irreversible adsorption of the therapeutic agent into the medical device. In addition, typically therapeutic agents are aqueous-based, whereas transfection agents are organic-based. Thus, it is difficult to combine the two without forming an undesirable emulsion, as the solvents used in each formulation may be incompatible.

Accordingly, there is a need for a more efficient method of delivering a therapeutic agent to targeted body tissue. There is also a need for an efficient method of applying costly therapeutic agents to a medical device surface, and for such method that will not adversely affect the formulation comprising the therapeutic agent that is to be applied to the medical device. There is also need a for a medical device made by such methods.

### SUMMARY OF THE INVENTION

These and other objectives are accomplished by the present invention. The present invention provides a coated medical device for delivering a therapeutic agent to body tissue. The coated medical device comprises a medical device having a surface suitable for exposure to body tissue, a first coating layer adhering to at least a portion of the surface of the medical device, and a second coating layer disposed over at least a portion of the first coating layer. The first coating layer comprises a transfection agent, and the second coating layer comprises a therapeutic agent to be delivered to the body tissue. The transfection agent is different from the therapeutic agent, and the first coating layer is substantially free of the therapeutic agent, and the second coating layer is substantially free of the transfection agent. In addition, the transfection agent enhances the delivery of the therapeutic agent to the body tissue, and reduces the surface tension of the surface so that adhesion of the second coating layer to the surface is enhanced as compared to when the first coating layer is absent. The transfection agent may be a poloxamer and the therapeutic agent may be a genetic material.

In an alternate embodiment, a coated medical device comprises a medical device having a surface suitable for exposure to body tissue; a first coating layer adhering to at least a portion of the surface; and a second coating layer disposed over at least a portion of the first coating layer. In this embodiment, the first coating layer comprises a poloxamer and the second coating layer comprises a genetic material to be delivered to the body tissue. The first coating layer is substantially free of the genetic material, and the second coating layer is substantially free of the poloxamer. Also, the poloxamer enhances the delivery of the genetic material to the body tissue and reduces the surface tension of the surface so that adhesion of the second coating layer to the surface is enhanced as compared to when the first coating layer is absent.

In another embodiment, a method of making a coated medical device is disclosed. The method of the present invention comprises providing a medical device having a surface suitable for exposure to body tissue; coating at least a portion of the surface of the medical device with a first coating layer, wherein the first coating layer comprises a transfection agent; and coating at least a portion of the first coating layer with a second coating layer, wherein the second coating layer comprises a therapeutic agent to be delivered to the body tissue. The transfection agent is different from the therapeutic agent, and the first coating layer is substantially free of the therapeutic agent, and the second coating layer is substantially free of the transfection agent. The transfection agent enhances the delivery of the therapeutic agent to the body tissue and reduces the surface tension of the surface so that adhesion of the second coating layer to the surface is enhanced as compared to when the first coating layer is absent. The transfection agent may be a poloxamer, and the therapeutic agent may be a genetic material.

The present invention provides an efficient method of applying a costly therapeutic agent to a medical device. The transfection agent in the first coating layer decreases the surface tension of the hydrophobic surface of the medical device which in turn increases the affinity of a therapeutic agent, which is in an aqueous solution, to the surface of the medical device. The second coating layer, which comprises the therapeutic agent, adheres to the surface of the medical device better than it would if the first coating layer were not present. Because a relatively inexpensive first coating layer is applied to the surface of the medical device to reduce the surface tension and improve adhesion of the more costly therapeutic agent thereon, the present method is less costly and less therapeutic agent is wasted during the coating process.

The present invention also provides a more efficient method of delivering therapeutic agent such as genetic materials to targeted body tissue. Having the transfection agent and the therapeutic agent in separate coating layers as opposed to having both in the same coating layer reduces the risk of interference between the transfection agent and the therapeutic agent and inactivation of the therapeutic agent during formulation of the two layers. In addition to providing suitable wettability for the second coating layer, the transfection agent enhances the transfection of the therapeutic agent to the targeted cells of the body lumen.

### BRIEF DESCRIPTION OF THE DRAWINGS

FIG. 1 is a chart showing the effectiveness of the first coating layer of the present invention in promoting the adherence of the second coating layer to the medical device surface.

### DESCRIPTION OF THE PREFERRED EMBODIMENTS

The medical devices appropriate for use in the present invention have a surface that is suitable for exposure to body tissue. Suitable medical devices include, but are not limited to, stents, surgical staples, catheters, such as central venous catheters and arterial catheters, guidewires, cannulas, cardiac pacemaker leads or lead tips, cardiac defribrillator leads or lead tips, GDC coils, implantable vascular access ports, blood storage bags, blood tubing, vascular or other grafts, intra-aortic balloon pumps, heart valves, cardiovascular sutures, total artificial hearts and ventricular assist pumps, extra-corporeal devices such as blood oxygenators, blood filters, hemodialysis units, hemoperfusion units, or plasmapheresis units.

Medical devices which are particularly suitable for the present invention include any stent for medical purposes, which are known to the skilled artisan. Suitable stents include, for example, vascular stents such as self-expanding stents and balloon expandable stents. Examples of self-expanding stents are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al. The framework of the suitable stents may be formed through various methods as known in the art. The framework may be welded, molded, laser cut, electro-formed, or consist of filaments or fibers which are wound or braided together in order to form a continuous structure.

The medical devices suitable for the present invention may be fabricated from polymeric and/or metallic materials. Suitable polymeric materials include without limitation polyurethane and its copolymers, silicone and its copolymers, ethylene vinyl-acetate, polyethylene terephthalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646.

The first coating layer, which is disposed on at least a portion of the surface of the medical device, is preferably biocompatible. The first coating layer is also preferably biodegradable. For example, the first coating layer may include a biological activator such as, but is not limited to, biodegradable polymers, elastomers, and oligomers of such monomers as caprolactone, lactic and glycolic acid, hydroxybutyrate, trimethylene carbonate, amino acids, anhydrides, or any variety of vinyl alcohols, biocompatible salts, proteins such as albumin, collagen, elastin or ECM matrices, dextran, and heparin, ionic and non-ionic surfactants that can be effective transfection agents, cationic lipids, polyethyleneimines, and chitosan.

Generally, the first coating layer comprises a component that both reduces the surface tension of the surface of the medical device while also enhancing the delivery of the therapeutic agent in the second coating layer to body tissue. For example, if the therapeutic agent is a protein, the first coating layer may be a conformation stabilizer such as albumin. When the therapeutic agents are cells, the first coating layer may be an extracellular matrix scaffold. If the therapeutic agent is a gene or genetic material, the first coating layer may be a transfection agent that enhances transfection of genetic material such as plasmid DNA.

Preferably, the first coating layer comprises a transfection agent. Preferably, the transfection agent is biodegradable. The transfection agent is preferably a relatively inexpensive, biocompatible material that promotes the adhesion of the second coating layer to the surface of the medical device, particularly during the coating process. More particularly, the transfection agent reduces the surface tension of the surface of the medical device so that adhesion of the second coating layer is enhanced as compared to when the first coating layer is absent.

The transfection agent also enhances the delivery of the therapeutic agent to the targeted body tissue. Thus, the transfection agent used in the present invention makes it more efficient to apply aqueous solutions or dispersions of therapeutic agents onto a stent while enhancing transfection of the therapeutic agent into the targeted body tissue.

Suitable transfection agents include, but are not limited to, poloxamers, dendrimers, polyvinyl pyrrolidone, liposomes, chitosan, calcium, and glycerol. Preferred transfection agents include, but are not limited to, poloxamers and dendrimers. Poloxamers are surfactants. A poloxamer is a series of copolymers composed of polyoxyethylene and polyoxypropylene blocks. The poloxamers vary in total molecular weight, polyoxypropylene to polyoxyethylene ratio, and surfactant properties.

Dendrimers are three-dimensional, highly ordered oligomeric compounds and polymeric compounds, respectively, which form on the basis of an initiator core that has several reactive groups. Substances are attached to these groups. Dendrimers of the first generation are obtained in this way. It is possible to bind to the substances of the first-generation dendrimers further substances and/or further initiator cores that can then be linked with further substances. In this connection, dendrimers of the second generation are obtained. Dendrimers of higher generations are obtained when this reaction sequence is repeated.

The second coating layer that is disposed over at least a portion of the first coating layer, can partially or fully cover the first coating layer. The second coating layer comprises a therapeutic agent to be delivered to body tissue.

The term “therapeutic agent” encompasses therapeutics, such as drugs, and also genetic materials and biological materials. Preferably, the therapeutic agent is a genetic material. Suitable genetic materials include, but are not limited to, DNA or RNA, such as, without limitation, DNA/RNA encoding a useful protein, RNA interference sequences (either encoded in DNA or RNA form), and DNA/RNA intended to be inserted into a human body including viral vectors and non-viral vectors. Suitable viral vectors include adenoviruses, gutted adenoviruses, adeno-associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, sketetal myocytes, macrophage), replication competent viruses (e.g., ONYX-015), and hybrid vectors. Suitable non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD).

Suitable biological materials also include cells, yeasts, bacteria, proteins, peptides, cytokines and hormones. Examples of suitable peptides and proteins include growth factors (e.g., FGF, FGF-1, FGF-2, VEGF, Endotherial Mitogenic Growth Factors, and epidermal growth factors, transforming growth factor α and β, platelet derived endothelial growth factor, platelet derived growth factor, tumor necrosis factor α, hepatocyte growth factor and insulin like growth factor ), transcription factors, proteinkinases, CD inhibitors, thymidine kinase, and bone morphogenic proteins (BMP's), such as BMP-2, BMP-3, BMP-4, BMP-5, BMP-6 (Vgr-1), BMP-7 (OP-1), BMP-8. BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xcnogeneic), genetically engineered, if desired, to deliver proteins of interest at the transplant site. The delivery media can be formulated as needed to maintain cell function and viability. Cells include whole bone marrow, bone marrow derived mono-nuclear cells, progenitor cells (e.g., endothelial progenitor cells) stem cells (e.g., mesenchymal, hematopoietic, neuronal), pluripotent stem cells, fibroblasts, macrophage, and satellite cells.

Therapeutic agent also includes non-genetic therapeutic agents, such as: anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid, amlodipine and doxazosin; anti-inflammatory agents such as glucocorticoids, betamethasone, dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides; vascular cell growth promotors such as growth factors, Vascular Endothelial Growth Factors (FEGF, all types including VEGF-2), growth factor receptors, transcriptional activators, and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents, vasodilating agents, and agents which interfere with endogenous vasoactive mechanisms; anti-oxidants, such as probucol; antibiotic agents, such as penicillin, cefoxitin, oxacillin, tobranycin; heme oxygenase; serine protease inhibitors; enos, inos (nitric oxide synthesis); angiogenic substances, such as acidic and basic fibrobrast growth factors, estrogen including estradiol (E2), estriol (E3) and 17-Beta Estradiol; and drugs for heart failure, such as digoxin, beta-blockers, angiotensin-converting enzyme (ACE) inhibitors including captopril and enalopril.

Other therapeutic agents include anti-proliferative drugs such as steroids, vitamins, and restenosis-inhibiting agents. Preferred restenosis-inhibiting agents include microtubule stabilizing agents such as Taxol, paclitaxel, paclitaxel analogues, derivatives, and mixtures thereof. For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

Other therapeutic agents also include nitroglycerin, nitrous oxides, antiobitics, aspirins, digitalis, and glycosides.

The therapeutic agent is different from the transfection agent, and the first coating layer is substantially free of the therapeutic agent, and the second coating layer is substantially free of the transfection agent. Preferably, the transfection agent is a poloxamer and the therapeutic agent is a genetic material.

The amount of the therapeutic agent present in the second coating layer can be adjusted to meet the needs of the patient. In general, the amount of the therapeutic agent used may vary depending on the application or therapeutic agent selected. In addition, the quantity of therapeutic agent used may be related to the selection of the transfection agent. One of skill in the art would understand how to adjust the amount of a particular therapeutic agent to achieve the desired dosage or amount.

The present invention also comprises a method of making a coated medical device described above. The method of making the medical device of the present invention comprises providing a medical device having a surface suitable for exposure to body tissue of a patient. At least a portion of the surface of the medical device is coated with a first coating layer comprising a transfection agent and coating at least a portion of the first coating layer with a second coating layer, wherein the second coating layer comprises a therapeutic agent. The transfection agent is preferably a poloxamer, and the therapeutic agent is preferably a genetic material.

Before applying each coating layer to the medical device, the coating materials of each coating layer should be dissolved or suspended in an aqueous or organic solvent. The solvent used with the first coating materials may be the same or different than the solvent used with the second coating materials. One or more solvents may be used with the coating materials of each coating layer. Suitable solvents are ones which can dissolve the coating materials into solution or form dispersions of the coating materials in the solvent. Any solvent which does not alter or adversely impact the properties of the therapeutic agent or the transfection agent can be employed in the method of the present invention. Examples of suitable solvents include, but are not limited to, tetrahydrofuran, methylethylketone, chloroform, toluene, acetonitrile, methylenechloride, water, aqueous buffers, and mixtures thereof.

For each coating layer, the coating materials are mixed together with the solvents to form a coating composition and then applied to the medical device. After the coating composition has been applied, the solvents are evaporated from the coating composition to form the coating layer.

The method of applying the coating layers generally involves applying a first coating layer material on the stent or medical device, allowing the stent to dry, and then applying the second coating layer material over the first coating layer, and then drying the stent. The coating layer materials are applied to the surface of the medical device by any suitable method as known by one skilled in the art. Suitable methods of applying the coating layer materials to the medical device include, but are not limited to, spray-coating, direct deposition such as by positive displacement or SLA, painting, rolling, electrostatic deposition, or a combination thereof. A preferred method is positive displacement or SLA.

The first coating layer and the second coating layer may be applied to the medical device by the same or different methods. The application of the first coating layer generally enhances the efficiency in applying the second coating layer using any technique. The first coating layer is stable under most coating conditions.

Coating layer materials may be applied one or more times to form a coating layer. For instance, several spray applications of the first coating material may be applied to the medical device surface to form the first coating layer. Generally, the total thickness of a coating layer is controlled by the number of applications of the coating material. The thickness ratio of the first coating layer to the second coating layer may be adjusted to obtain the desired effect.

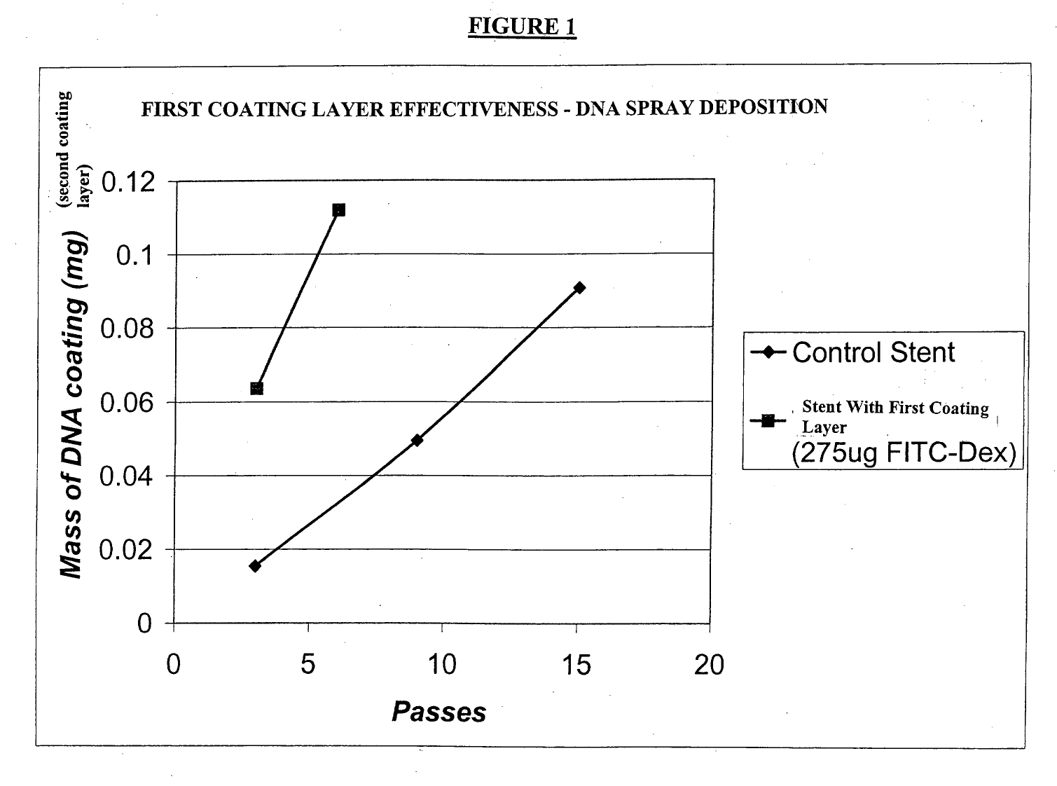

FIG. 1 shows the effectiveness of the first coating layer comprising a transfection agent in promoting the adherence of the second coating layer to the medical device surface. FIG. 1 shows that stents coated with a first coating layer (represented by the squares) required significantly fewer spray passes to coat the medical device surface with a given amount of therapeutic agent, in this case DNA, than stents lacking the first coating layer (the control, which is represented by the diamonds). In other words, the transfection agent of the first coating layer enhances the adhesion of the therapeutic agent in the second coating layer material to reduce the number of passes that would be required to deposit the same amount of DNA on a stent that is not coated with the first coating layer.

In use, a coated medical device, such as an expandable stent, according to the present invention can be made to provide a desired release profile of the therapeutic agent. The medical devices and stents of the present invention may be used for any appropriate medical procedure. Delivery of the medical device can be accomplished using methods well known to those skilled in the art, such as mounting the stent on an inflatable balloon disposed at the distal end of a delivery catheter.

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

What is claimed is:

1. A coated medical device comprising:
a medical device having a surface suitable for exposure to body tissue;
a first coating layer adhering to at least a portion of the surface, wherein the first coating layer comprises a transfection agent; and
a second coating layer disposed over at least a portion of the first coating layer, wherein the second coating layer comprises a therapeutic agent to be delivered to the body tissue,
wherein the transfection agent is different from the therapeutic agent, wherein the first coating layer is substantially free of the therapeutic agent, and the second coating layer is substantially free of the transfection agent, and wherein the transfection agent enhances the delivery of the therapeutic agent to the body tissue, and reduces the surface tension of the surface so that adhesion of the second coating layer to the surface is enhanced as compared to when the first coating layer is absent.

2. The medical device of claim 1, wherein the transfection agent is a poloxamer.

3. The medical device of claim 1, wherein the therapeutic agent is a genetic material.

4. The medical device of claim 1, wherein the therapeutic agent is hydrophilic.

5. The medical device of claim 1, wherein the medical device is a stent.

6. A coated medical comprising:
a medical device having a surface suitable for exposure to body tissue;
a first coating layer adhering to at least a portion of the surface, wherein the first coating layer comprises a poloxamer; and
a second coating layer disposed over at least a portion of the first coating layer, wherein the second coating layer comprises a genetic material to be delivered to the body tissue,
wherein the first coating layer is substantially free of the genetic material, and the second coating layer is substantially free of the poloxamer, and wherein the poloxamer enhances the delivery of the genetic material to the body tissue and reduces the surface tension of the surface so that adhesion of the second coating layer to the surface is enhanced as compared to when the first coating layer is absent.

7. The medical device of claim 6, wherein the medical device is a stent.

8. A method of making a coated medical device for delivering a therapeutic agent to body tissue comprising:
providing a medical device having a surface suitable for exposure to body tissue;
coating at least a portion of the surface with a first coating layer, wherein the first coating layer comprises a transfection agent; and
coating at least a portion of the first coating layer with a second coating layer, wherein the second coating layer comprises a therapeutic agent to be delivered to the body tissue,
wherein the transfection agent is different from the therapeutic agent, wherein the first coating layer is substantially free of the therapeutic agent, and the second coating layer is substantially free of the transfection agent, and wherein the transfection agent enhances the delivery of the therapeutic agent to the body tissue and reduces the surface tension of the surface so that adhesion of the second coating layer to the surface is enhanced as compared to when the first coating layer is absent.

9. The method of claim 8, wherein the first coating layer is applied by spraying.

10. The method of claim 8, wherein the second coating layer is applied by a technique selected from the group consisting of spraying, dipping, and direct deposition.

11. The method of claim 8, wherein the transfection agent is a poloxamer.

12. The method of claim 8, wherein the therapeutic agent is a genetic material.

13. The method of claim 8, wherein the transfection agent is a poloxamer and the therapeutic agent is a genetic material.

14. The method of claim 8, wherein the medical device is a stent.
